(19) United States
(12) Patent Application Publication
(10) Pub. No.: US 2018/0221666 A1
(43) Pub. Date: Aug. 9, 2018

(54) SYSTEMS AND METHODS FOR OPTIMIZING TARGETED NEUROPLASTICITY

(71) Applicant: THE BOARD OF REGENTS OF THE UNIVERSITY OF TEXAS SYSTEM, Austin, TX (US)

(72) Inventors: ROBERT L. RENNAKER, II, Sachse, TX (US); MICHAEL P. KILGARD, Richardson, TX (US); SETH HAYS, Richardson, TX (US)

(73) Assignee: THE BOARD OF REGENTS OF THE UNIVERSITY OF TEXAS SYSTEM, Austin, TX (US)

(21) Appl. No.: 15/590,368

(22) Filed: May 9, 2017

Related U.S. Application Data
(60) Provisional application No. 62/333,982, filed on May 10, 2016.

(51) Int. Cl. A61N 1/36

(52) U.S. Cl. CPC A61N 1/36092; A61N 1/36132; A61N 1/36167; A61N 1/36053

(57) ABSTRACT

Systems and methods for optimizing targeted neuroplasticity training. Exemplary embodiments include a vagus nerve stimulator, a controller, and a monitoring device.

### CROSS-REFERENCE TO RELATED APPLICATIONS

This application claims priority to U.S. Provisional Patent Application Ser. No. 62/333,982 filed May 10, 2016, the entire contents of which are incorporated by reference herein.

### GOVERNMENT LICENSE RIGHTS

This invention was made with government support under R01 NS085167 by the National Institutes of Health and N66001-15-2-4057 by the Office of Naval Research. The government has certain rights in the invention.

### BACKGROUND INFORMATION

Mastery of a complex skill, like learning a language, requires thousands of hours of practice. Despite improvements in education techniques over the past century, learning rates for these skills remains largely unchanged. Recent developments in neuroscience indicate that precisely timed peripheral nerve stimulation can engage brain regions that regulate learning and could greatly accelerate learning rates.

Neural plasticity is the basis for learning. Therefore, techniques that can guide robust, specific, and long-lasting neuroplasticity hold promise to accelerate acquisition of complex skills, such as foreign language learning, a golf swing, or even operating advanced technologies. Over the last forty years, the factors that regulate neural plasticity have been elucidated. Pioneering neuroscience studies demonstrated that even slight differences in the timing of neural activity can impact whether synaptic connections are strengthened or weakened [20]. Moreover, the timing and level of neuromodulator release interacts with neural activity to dictate the magnitude and polarity of plasticity [21, 22].

### SUMMARY

Exemplary embodiments of the present disclosure comprise disruptive technologies that enhance the neural plasticity responsible for learning. Brief pulses of vagus nerve stimulation (VNS) drive release of plasticity-promoting neuromodulators in the brain that serve to facilitate robust, specific changes in neural circuitry [1-6]. Research demonstrates that VNS delivery paired in time with rehabilitation enhances plasticity to accelerate recovery of normal function after nervous system damage [3, 7-15]. The inventors have successfully guided the development of VNS-enhanced neurorehabilitation from preclinical proof-of-concept to clinical evaluation in multiple neurological disorders [14-19].

Exemplary embodiments may comprehensively characterize VNS-dependent plasticity across the parameter space to identify paradigms that differentially control plasticity. In addition, exemplary embodiments may demonstrate that VNS-based targeted neuroplasticity training (TNT) can enhance both motor and sensory learning such as what is needed for speech processing.

Exemplary embodiments use brief pulses of vagus nerve stimulation (VNS) to drive release of plasticity-promoting neuromodulators that serve to facilitate robust, specific changes in neural circuitry [1-6,23-27]. The inventors have demonstrated that VNS paired with rehabilitative training enhances recovery in preclinical models of sensory, motor, and affective disorders [3,7-15,28]. Moreover, the inventors have guided the development of this targeted plasticity therapy to four clinical trials in patients in five years [14-19]. The inventors' most recent clinical study indicates that VNS-based targeted plasticity therapy triples the benefits of rehabilitation in stroke patients [14], highlighting the potential of this strategy to enhance adaptive neuroplasticity in injured and healthy humans.

Using these same principles of guided neuroplasticity, VNS paired with training represents a promising method to accelerate learning of complex skills. Precise control of plasticity may be required to optimally accelerate complex learning. Therefore, it is beneficial to define VNS paradigms that allow selective potentiation and depression of neural circuits to increase the dynamic range of accelerated learning.

Landmark experiments over the last forty years have demonstrated that the timing of neural activity is a critical determinant in both the magnitude and the polarity of synaptic plasticity. Minor changes in the relative timing of neural activity strongly influence the strengthening or weakening of synaptic connections [20,29]. When an action potential in a presynaptic neuron precedes depolarization of the postsynaptic neuron, the connection between the neurons is strengthened. The closer the neural activity is in time, the stronger the magnitude of strengthening. Conversely, when postsynaptic depolarization precedes a presynaptic action potential, the connection between the neurons is weakened as a function of the temporal proximity This process, referred to as spike-timing-dependent plasticity (STDP), accounts for activity-dependent long-term potentiation (LTP) and long-term depression (LTD) in neural circuits [20].

Additional experiments have expanded the understanding of this process and demonstrated that neuromodulators regulate the timing rules of STDP [21,22]. Neuromodulators influence signaling cascades in part by activating G-protein-coupled receptors (GPCRs). In turn, these receptors transduce intracellular signaling through coupling with adenylyl cyclase (AC) and phospholipase C (PLC) to mediate plasticity. Activation of PLC alters the timing rules to preferentially favor LTD, while activation of AC alters the rules to favor LTP [21]. The relative levels AC and PLC activation, in conjunction with the temporal sequence of neural activity, interact to yield synaptic LTP or LTD [21,30]. Moreover, recent evidence demonstrates that the timing of neuromodulator release relative to neural activity exerts a strong influence on strengthening or weakening of synaptic connections [22].

In addition to the relative timing of neural activity, the GPCRs that mediate neuromodulator function exhibit features that affect function over time. GPCRs desensitize in a concentration-dependent manner, such that strong stimulation results in greater desensitization that reduces receptor availability and blunts subsequent activation [31]. Moreover, GPCRs are homeostatically regulated based on previous activation [31,32]. As a result, repeated strong activations can yield a downregulation in receptor levels, thus weakening future responses.

Elucidation of the principles of synaptic plasticity at the cellular level has provided important insight into the factors that regulate learning; however, an understanding of synaptic plasticity alone is not sufficient to predict network plasticity and consequently meaningful functional changes for individuals. This disclosure leverage insights into synaptic plasticity principles and empirically evaluate VNS parameters to specifically drive circuit-level changes and ultimately influence the rate of learning of motor, sensory and cognitive tasks. The inventors' preliminary studies provide compelling evidence that the parameters of VNS pairing strongly influence the effects on synaptic plasticity. These findings, demonstrate that the stimulation parameters (rate, amplitude, and pulse width), as well as the relative timing to behavior significantly alter the effect on behavioral consequence. The stimulation parameters can selectively depress or potentiate cortical circuits. This ability to strengthen and weaken neural networks represents a powerful method to tune neural plasticity to treat disease and enhance learning.

Language learning is a complex skill that involves plasticity in multiple brain systems, including the auditory cortex for receptive speech and the motor cortex for productive speech [33,34]. The auditory and motor systems exhibit substantially different temporal coding of information. In the auditory system, the event (sound) precedes neural activity. In the motor system, neural activity precedes the event (movement of the lips, jaw, and tongue). The distinct differences in timing of neural activity suggest that VNS may have difference consequences on plasticity in the auditory and motor systems. While much more complex, the higher cognitive functions to associate these sounds with objects operate using the same rules as the motor and sensory system. Therefore, in order to maximize the efficacy of learning, it is desirable to define paradigms that allow selective potentiation and depression within neural circuits responsible for learning or enhancing motor, sensory and cognitive function.

The use of closed-loop stimulation can be used to drive differing changes in the neural circuitry. For example, stimulation just prior to exposure of a stimulus that elicits fear in individuals with post-traumatic stress could weaken those connections that drive the fear response. While stimulation paired or just following the same stimulus would result in an enhancement in the fear response. VNS can, in effect accelerate learning a skill as well as unlearning or new learning. This ability could have applications for multiple clinical conditions including addictive behaviors, fear, anxiety and a host of other neuropsychological conditions.

While timing of VNS relative to a skill learning task is important, changes in the stimulation parameters can have a similar effect. Stimulation at higher rates (>60 Hz) can accelerate unlearning (weaken neural connections), while stimulation at lower rates can accelerate learning (strengthen neural connections).

By combining the stimulation parameters as well as the coupling with skill learning, an optimum paradigm can be selected for accelerating learning or unlearning.

Certain embodiments include a closed loop system for optimizing learning, the system comprising: a vagus nerve stimulator configured to stimulate a vagus nerve of a person with an electrical pulse train; a controller configured to alter one or more parameters of the electrical pulse train while maintaining other parameters of the electrical pulse train constant; and a monitoring device configured to monitor skill learning, wherein the controller receives feedback from the monitoring device and alters the parameters of the electrical pulse train of the vagus nerve stimulator. In some embodiments the vagus nerve stimulator is configured to stimulate a vagus nerve of a person with an electrical pulse train prior to the person performing a process related to the skill learning. In certain embodiments the vagus nerve stimulator is configured to stimulate a vagus nerve of a person with an electrical pulse train for a period of 1-10 seconds prior to the person performing a process related to the skill learning. In particular embodiments the vagus nerve stimulator is configured to stimulate a vagus nerve of a person with an electrical pulse train for a period of 1-5 seconds prior to the person performing a process related to the skill learning. In some embodiments the vagus nerve stimulator is configured to stimulate a vagus nerve of a person with an electrical pulse train after the person performs a process related to the skill learning. In specific embodiments the vagus nerve stimulator is configured to stimulate a vagus nerve of a person with an electrical pulse train for a period of 1-10 seconds after the person performs a process related to the skill learning. In certain embodiments the vagus nerve stimulator is configured to stimulate a vagus nerve of a person with an electrical pulse train for a period of 1-5 seconds after the person performs a process related to the skill learning.

In particular embodiments the one or more parameters of the electrical pulse train altered by the controller include electrical pulse train duration and electrical pulse train frequency; and the parameters of the electrical pulse train maintained constant include pulses per electrical pulse train, current intensity, and total charge. In some embodiments the electrical pulse train duration is altered between 0.125 seconds, 0.5 seconds and 2.0 seconds. In specific embodiments the electrical pulse train frequency is altered between 7.5 Hz, 30 Hz, and 120 Hz. In certain embodiments the one or more parameters of the electrical pulse train altered by the controller include pulses per electrical pulse train, electrical pulse train frequency, and total charge; and the parameters of the electrical pulse train maintained constant include current intensity. In some embodiments the pulses per electrical pulse train is altered between 4 pulses, 16 pulses and 64 pulses. In particular embodiments the electrical pulse train frequency is altered between 7.5 Hz, 30 Hz, and 120 Hz. In specific embodiments the one or more parameters of the electrical pulse train altered by the controller include electrical pulse train duration, pulses per electrical pulse train, and total charge; and the parameters of the electrical pulse train maintained constant include current intensity and electrical pulse train frequency. In certain embodiments the pulses per electrical pulse train is altered between 4 pulses, 16 pulses and 64 pulses. In some embodiments the electrical pulse train duration is altered between 0.125 seconds, 0.5 seconds and 2.0 seconds. In particular embodiments the one or more parameters of the electrical pulse train altered by the controller include current intensity, electrical pulse train frequency, and pulses per electrical pulse train; and the parameters of the electrical pulse train maintained constant include total charge and electrical pulse train duration. In specific embodiments the pulses per electrical pulse train is altered between 4 pulses and 16 pulses. In certain embodiments the electrical pulse train frequency is altered between 7.5 Hz and 30 Hz. In some embodiments the pulses per electrical pulse train is altered between 4 pulses and 16 pulses. In particular embodiments the vagus nerve stimulator is an implantable or transcutaneous device.

Certain embodiments include a method of optimizing targeted neuroplasticity training, the method comprising: stimulating a vagus nerve of a person with an electrical pulse train for a time period before or after the person performs a skill learning process; and monitoring the skill learning process before and after the vagus nerve is stimulated with the electrical pulse train. In some embodiments the time period is 1-10 seconds or 1-5 before the person performs the skill learning process. In particular embodiments the time period is 1-10 or 1-5 seconds after the person performs the skill learning process. Specific embodiments further comprise altering one or more parameters of the electrical pulse train while maintaining other parameters of the electrical pulse train constant. In certain embodiments altering one or more parameters of the electrical pulse train while maintaining other parameters of the electrical pulse train constant comprises: altering an electrical pulse train duration and an electrical pulse train frequency; and maintaining constant an electrical pulse train, current intensity, and total charge.

In particular embodiments the electrical pulse train duration is altered between 0.125 seconds, 0.5 seconds and 2.0 seconds.

In some embodiments the electrical pulse train frequency is altered between 7.5 Hz, 30 Hz, and 120 Hz. In specific embodiments altering one or more parameters of the electrical pulse train while maintaining other parameters of the electrical pulse train constant comprises: altering an electrical pulse train, electrical pulse train frequency, and total charge; and maintaining constant a current intensity. In certain embodiments the pulses per electrical pulse train is altered between 4 pulses, 16 pulses and 64 pulses. In some embodiments the electrical pulse train frequency is altered between 7.5 Hz, 30 Hz, and 120 Hz. In particular embodiments altering one or more parameters of the electrical pulse train while maintaining other parameters of the electrical pulse train constant comprises: altering an electrical pulse train duration, pulses per electrical pulse train, and total charge; and maintaining constant a current intensity and electrical pulse train frequency. In specific embodiments the pulses per electrical pulse train is altered between 4 pulses, 16 pulses and 64 pulses. In certain embodiments the electrical pulse train duration is altered between 0.125 seconds, 0.5 seconds and 2.0 seconds. In some embodiments altering one or more parameters of the electrical pulse train while maintaining other parameters of the electrical pulse train constant comprises: altering a current intensity, electrical pulse train frequency, and pulses per electrical pulse train; and maintaining constant a total charge and an electrical pulse train duration. In particular embodiments the electrical pulse train frequency is altered between 7.5 Hz and 30 Hz. In specific embodiments the pulses per electrical pulse train is altered between 4 pulses and 16 pulses.

In the present disclosure, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

The term “learning” is this strengthening of neural connections and or changes in behavior, skill, or perception.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE FIGURES

FIG. 1 illustrates a schematic of an exemplary embodiment according to the present disclosure.

FIG. 2 illustrates exemplary embodiments used in sensory training, motor training, and cognitive training.

FIG. 3 illustrates a graph of cortical response strength versus frequency for exemplary embodiments.

### DETAILED DESCRIPTION OF ILLUSTRATIVE EMBODIMENTS

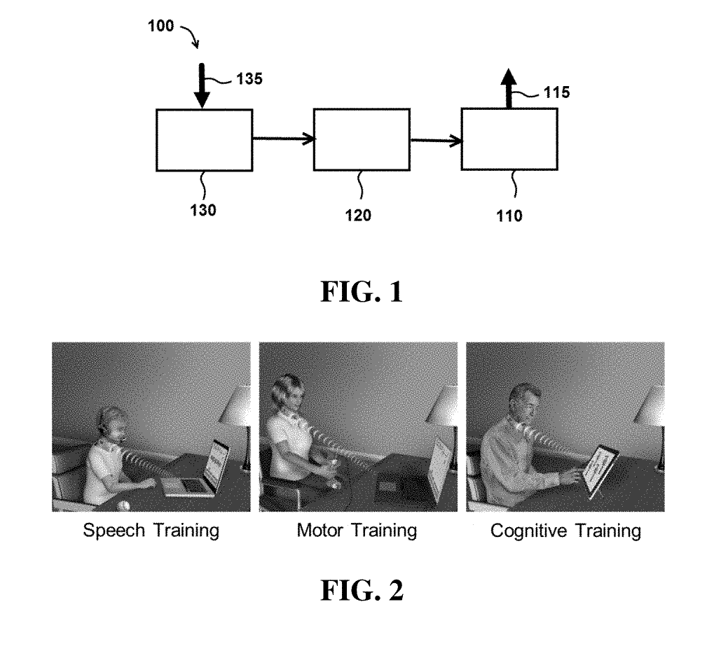

Referring now to FIG. 1, a schematic of a closed loop system 100 is shown for optimizing targeted neuroplasticity training In this embodiment, system 100 comprises a vagus nerve stimulator 110, a controller 120, and a monitoring device 130. During operation of system 100, vagus nerve stimulator 110 is configured to stimulate a vagus verve of a person with an electrical pulse train 115, and controller 120 is configured to alter one or more parameters of the electrical pulse train 115 while maintaining other parameters of the electrical pulse train constant. In addition, monitoring device 130 is configured to monitor a neural network response 135 before and after the vagus nerve is stimulated by vagus nerve stimulator 110. In certain embodiments, monitoring device 130 can be configured to monitor plasticity in the auditory and/or motor cortices.

In particular embodiments, controller 120 receives feedback from monitoring device 130 and alters the parameters of the electrical pulse train of vagus nerve stimulator 110.

In certain embodiments, vagus nerve stimulator 110 may comprise a pair of electrodes, including for example, cuff electrodes, conductive plates or other suitable configurations.

Vagus nerve stimulator 110 may be powered by a piezoelectric, near field inductive power transfer, far-field inductive power transfer, battery, rechargeable battery or other suitable neurostimulator power system. When vagus nerve stimulator 110 receives input from controller 120, vagus nerve stimulator 110 can initiate a stimulation pulse to the vagus nerve via the electrodes.

In certain embodiments, vagus nerve stimulator 110 can provide stimulation either before or after a skill learning process takes place in order to promote “learning” or “unlearning”. For example, vagus nerve stimulator 110 may provide stimulation for a time period (e.g. 1-5 seconds or 1-10 seconds) after a skill learning process is performed by the person to accelerate or promote the learning of the skill.

In other embodiments, vagus nerve stimulator 110 may provide stimulation for a time period (e.g. 1-5 seconds or 1-10 seconds) before an event to promote “unlearning.” For example, vagus nerve stimulator 110 may provide stimulation before a person is exposed to a stimulus that elicits fear in individuals with post-traumatic stress, anxiety or other neuropsychological conditions.

Exemplary embodiments of the present disclosure represent a transformative platform technology that can apply broadly to learning. Pairing sensory, motor, and cognitive training regimens with VNS has the potential to accelerate the acquisition of a broad range of complex skills. Inventors' studies indicate that targeted plasticity technologies are effective in the context of a wide variety of pathologies and across the lifespan [3,7-15,28]. The principles derived from these studies can apply broadly to learning of complex skills to optimize human aptitude including motor, sensory and cognitive functions. As shown in FIG. 2, embodiments of system 100 can be used in sensory training, motor training, and cognitive training.

Particular embodiments of the present disclosure can be used to identify VNS paradigms/parameters that specifically promote cortical map expansion and contraction. Certain embodiments systematically vary parameters of stimulation provided by vagus nerve stimulator 110 and evaluate the effects on plasticity in the auditory and motor cortices via monitoring device 130. Because some parameters are inherently dependent upon one another (i.e., frequency, number of pulses per train, and train duration cannot be independently varied), exemplary embodiments can be configured to vary a subset of parameters while keeping others constant. Evaluating various interactions illustrates parameters that are important to mediate plasticity.

By documenting the effects of these stimulation paradigms on cortical plasticity, parameter sets provided by controller 120 for vagus nerve stimulator 110 that drive maximal depression and potentiation in circuits required for productive and receptive language can be identified. Exemplary embodiments can assess plasticity to a wide range of parameters in one domain (auditory or motor) and confirm these findings in the other domain using a sub-selection of the parameter sets that drive the maximal differences in plasticity.

Exemplary embodiments can demonstrate that VNS paired with training can drive robust, specific map expansion in the auditory and motor cortices. While previous studies with standard parameters drive VNS-dependent expansion, no reversible manipulations currently exist to engender contraction of cortical maps. The ability to bidirectionally control plasticity by selectively driving map expansion and contraction can allow greater dynamic range to precisely tune neural circuits.

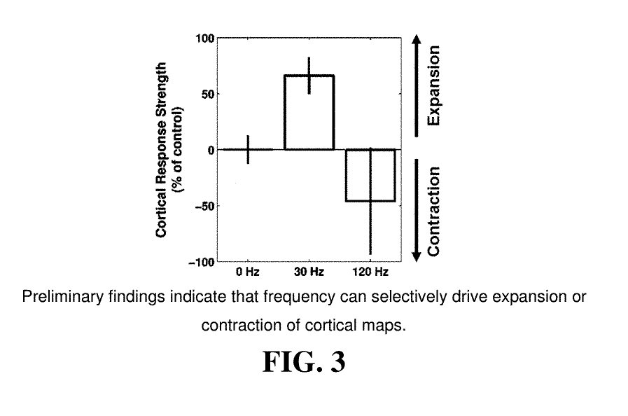

Data obtained using exemplary embodiments is illustrated in FIG. 3. This data indicates that the frequency of pulses during VNS stimulation trains allows selective potentiation or depression of map area. This differential plasticity is likely a product of changes in timing of activity and neuromodulatory release and reflects both pre- and postsynaptic mechanisms. Empirical determination of parameters that can selectively depress or potentiate cortical networks represents a potentially powerful method to tune neural plasticity and enhance learning.

Learning occurs through a process of strengthening some neural connections and weakening others. Prior uses of VNS to enhance learning have focused exclusively on strengthening. These results suggest that it may be possible to optimize learning using VNS by pairing certain events with low frequency stimulation and other events with high frequency stimulation.

For example, particular muscles are overly tense to allow for fluid movement during skilled actions. Pairing activity in the muscle of interest with high frequency VNS is expected to reduce the tension on the muscle and improve task performance.

Sensory responses are often pathologically strong. Hyperacusis, for example, is the perception that sounds are painfully loud when the intensity is not disturbing to most people. Pairing sounds with high frequency VNS could be useful for weakening the response to stimuli that evoke an overactive response. Pairing low frequency VNS with sounds that do not evoke a hyperactive response would be expected so shift the neural network to favor more useful sensory responsiveness.

For cognitive and emotional tasks, many individuals perform because certain stimuli are distracting or evoke an exaggerated emotional response. Pairing high frequency VNS with experiences that are overly distracting or arousing and pairing low frequency VNS with experiences that need to be learned is expected to yield improved learning over either approach in isolation.

All of the devices, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The contents of the following references are incorporated by reference herein:

### REFERENCES

1. C. T. Engineer, N. D. Engineer, J. R. Riley, J. D. Seale, M. P. Kilgard, Pairing Speech Sounds With Vagus Nerve Stimulation Drives Stimulus-specific Cortical Plasticity. Brain Stimulation. (2015).

2. M. S. Borland, W. A. Vrana, N. A. Moreno, E. A. Fogarty, E. P. Buell, P. Sharma, C. T. Engineer, M. P. Kilgard, Cortical Map Plasticity as a Function of Vagus Nerve Stimulation Intensity. Brain Stimulation. (In press).

3. N. D. Engineer, J. R. Riley, J. D. Seale, W. A. Vrana, J. A. Shetake, S. P. Sudanagunta, M. S. Borland, M. P. Kilgard, Reversing pathological neural activity using targeted plasticity. Nature. 470, 101-104 (2011).

4. D. R. Hulsey, S. A. Hays, N. Khodaparast, A. Ruiz, P. Das, R. L. Rennaker, M. P. Kilgard, Reorganization of Motor Cortex by Vagus Nerve Stimulation Requires Cholinergic Innervation. Brain Stimulation. (2016).

5. B. A. Porter, N. Khodaparast, T. Fayyaz, R. J. Cheung, S. S. Ahmed, W. A. Vrana, R. L. Rennaker II, M. P. Kilgard, Repeatedly Pairing Vagus Nerve Stimulation with a Movement Reorganizes Primary Motor Cortex. Cerebral Cortex. 22, 2365-2374 (2011).

6. J. A. Shetake, N. D. Engineer, W. A. Vrana, J. T. Wolf, M. P. Kilgard, Pairing tone trains with vagus nerve stimulation induces temporal plasticity in auditory cortex. Exp. Neurol. 233, 342-49 (2011).

7. S. A. Hays, R. L. Rennaker II, M. P. Kilgard, Targeting plasticity with vagus nerve stimulation to treat neurological disease. Prog. Brain Res. 207, 275-299 (2013).

8. S. A. Hays, N. Khodaparast, A. Ruiz, A. M. Sloan, D. R. Hulsey, R. L. Rennaker, M. P. Kilgard, The timing and amount of vagus nerve stimulation during rehabilitative training affect post-stroke recovery of forelimb strength. NeuroReport. 25(9), 676-682 (2014).

9. N. Khodaparast, S. A. Hays, A. M. Sloan, T. Fayyaz, D. R. Hulsey, R. L. Rennaker II, M. P. Kilgard, Vagus nerve stimulation delivered during motor rehabilitation improves recovery in a rat model of stroke. Neurorehabil. Neural Repair. 28, 698-706 (2014).

10. D. Pruitt, A. Schmid, L. Kim, C. Abe, J. Trieu, C. Choua, S. Hays, M. Kilgard, R. L. Rennaker II, Vagus nerve stimulation delivered with motor training enhances recovery of function after traumatic brain injury. J. Neurotrauma. (2015).

11. N. Khodaparast, M. P. Kilgard, R. Casavant, A. Ruiz, I. Qureshi, P. D. Ganzer, R. L. Rennaker 2nd, S. A. Hays, Vagus Nerve Stimulation During Rehabilitative Training Improves Forelimb Recovery After Chronic Ischemic Stroke in Rats. Neurorehabil. Neural Repair. (2015).

12. N. Khodaparast, S. A. Hays, A. M. Sloan, D. R. Hulsey, A. Ruiz, M. Pantoja, R. L. Rennaker II, M. P. Kilgard, Vagus nerve stimulation during rehabilitative training improves forelimb strength following ischemic stroke. Neurobiol. Dis. 60, 80-88 (2013).

13. S. A. Hays, N. Khodaparast, D. R. Hulsey, A. Ruiz, A. M. Sloan, R. L. Rennaker II, M. P. Kilgard, Vagus Nerve Stimulation during Rehabilitative Training Improves Functional Recovery after Intracerebral Hemorrhage. Stroke. 45, 10-3097-3100 (2014).

14. J. Dawson, D. Pierce, A. Dixit, T. J. Kimberley, M. Robertson, B. Tarver, 0. Hilmi, J. McLean, K. Forbes, M. P. Kilgard, R. L. Rennaker, S. C. Cramer, M. Walters, N. Engineer, Safety, Feasibility, and Efficacy of Vagus Nerve Stimulation Paired With Upper-Limb Rehabilitation After Ischemic Stroke. Stroke. (2015).

15. D. De Ridder, S. Vanneste, N. D. Engineer, M. P. Kilgard, Safety and Efficacy of Vagus Nerve Stimulation Paired With Tones for the Treatment of Tinnitus: A Case Series. Neuromodulation: Technology at the Neural Interface. (2013).

16. Microtransponder, Paired Vagus Nerve Stimulation (VNS) With Rehabilitation for Upper Limb Function Improvement After Stroke. ClinicalTrials. gov. Bethesda (MD): National Library of Medicine (US). NCT01669161, https://clinicaltrials.gov/ct2/show/NCT1669161 (2014).

17. Microtransponder, Proof-of-Concept Study Assessing VNS Paired with Tones for Tinnitus. ClinicalTrials. gov. Bethesda (MD): National Library of Medicine (US). NCT01253616, Available from: http://clinicaltrials.gov/ct2/show/NCT01253616 (2010).

18. Microtransponder, Vagus Nerve Stimulation (VNS) Paired with Tones for Tinnitus. ClinicalTrials. gov. Bethesda (MD): National Library of Medicine (US). NCT01962558, http://clinicaltrials.gov/ct2/show/NCT01962558 (2013).

19. Microtransponder, VNS During Rehabilitation for Improved Upper Limb Motor Function After Stroke. ClinicalTrials. gov. Bethesda (MD): National Library of Medicine (US). NCT02243020, https://clinicaltrials.gov/ct2/show/study/NCT02243020 (2014).

20. Y. Dan, M. Poo, Spike timing-dependent plasticity of neural circuits. Neuron. 44, 23-30 (2004).

21. G. H. Seol, J. Ziburkus, S. Y. Huang, L. Song, I. T. Kim, K. Takamiya, R. L. Huganir, H. K. Lee, A. Kirkwood, Neuromodulators control the polarity of spike-timing-dependent synaptic plasticity. Neuron. 55, 919-929 (2007).

22. K. He, M. Huertas, S. Hong, X. Tie, J. Hell, H. Shouval, A. Kirkwood, Distinct Eligibility Traces for LTP and LTD in Cortical Synapses. Neuron. 88, 528-538 (2015).

23. L. Detari, G. Juhasz, T. Kukorelli, Effect of stimulation of vagal and radial nerves on neuronal activity in the basal forebrain area of anaesthetized cats. Acta Physiol. Hung. 61, 147-154 (1983).

24. A. E. Dorr, G. Debonnel, Effect of vagus nerve stimulation on serotonergic and noradrenergic transmission. J. Pharmacol. Exp. Ther. 318, 890-898 (2006).

25. R. W. Roosevelt, D. C. Smith, R. W. Clough, R. A. Jensen, R. A. Browning, Increased extracellular concentrations of norepinephrine in cortex and hippocampus following vagus nerve stimulation in the rat. Brain Res. 1119, 124-132 (2006).

26. S. E. Krahl, K. B. Clark, D. C. Smith, R. A. Browning, Locus coeruleus lesions suppress the seizure-attenuating effects of vagus nerve stimulation. Epilepsia. 39, 709-714 (1998).

27. J. Nichols, A. Nichols, S. Smirnakis, N. Engineer, M. Kilgard, M. Atzori, Vagus nerve stimulation modulates cortical synchrony and excitability through the activation of muscarinic receptors. Neuroscience. 189, 207-214 (2011).

28. D. F. Peñia, N. D. Engineer, C. K. McIntyre, Rapid Remission of Conditioned Fear Expression with Extinction Training Paired with Vagus Nerve Stimulation. Biological psychiatry. 73, 1071-1077 (2012).

29. H. Markram, J. Lubke, M. Frotscher, B. Sakmann, Regulation of synaptic efficacy by coincidence of postsynaptic APs and EPSPs. Science. 275, 213-215 (1997).

30. R. C. Malenka, M. F. Bear, LTP and LTD: an embarrassment of riches. Neuron. 44, 5-21 (2004).

31. R. R. Gainetdinov, R. T. Premont, L. M. Bohn, R. J. Lefkowitz, M. G. Caron, Desensitization of G protein-coupled receptors and neuronal functions. Annu. Rev. Neurosci. 27, 107-144 (2004).

32. S. K. Bohm, E. F. Grady, N. W. Bunnett, Regulatory mechanisms that modulate signalling by G-protein-coupled receptors. Biochem. J. 322 (Pt 1), 1-18 (1997).

33. D. E. Callan, K. Tajima, A. M. Callan, R. Kubo, S. Masaki, R. Akahane-Yamada, Learning-induced neural plasticity associated with improved identification performance after training of a difficult second-language phonetic contrast. Neuroimage. 19, 113-124 (2003).

34. C. Alain, J. S. Snyder, Y. He, K. S. Reinke, Changes in auditory cortex parallel rapid perceptual learning. Cereb. Cortex. 17, 1074-1084 (2007).

35. C. T. Engineer, C. A. Perez, Y. H. Chen, R. S. Carraway, A. C. Reed, J. A. Shetake, V. Jakkamsetti, K. Q. Chang, M. P. Kilgard, Cortical activity patterns predict speech discrimination ability. Nat. Neurosci. 11, 603-608 (2008).

36. C. T. Engineer, K. C. Rahebi, M. S. Borland, E. P. Buell, T. M. Centanni, M. K. Fink, K. W. Im, L. G. Wilson, M. P. Kilgard, Degraded neural and behavioral processing of speech sounds in a rat model of Rett syndrome. Neurobiol. Dis. 83, 26-34 (2015).

37. M. Kilgard, J. Vazquez, N. Engineer, P. Pandya, Experience dependent plasticity alters cortical synchronization. Hear. Res. 229, 171-179 (2007).

38. M. F. Bear, W. Singer, Modulation of visual cortical plasticity by acetylcholine and noradrenaline. (1986).

39. K. Clark, S. Krahl, D. Smith, R. Jensen, Post-training unilateral vagal stimulation enhances retention performance in the rat. Neurobiol. Learn. Mem. 63, 213-216 (1995).

40. K. B. Clark, D. K. Naritoku, D. C. Smith, R. A. Browning, R. A. Jensen, Enhanced recognition memory following vagus nerve stimulation in human subjects. Nat. Neurosci. 2, 94-98 (1999).

41. A. Reed, J. Riley, R. Carraway, A. Carrasco, C. Perez, V. Jakkamsetti, M. P. Kilgard, Cortical map plasticity improves learning but is not necessary for improved performance Neuron. 70, 121-131 (2011).

1. A closed loop system for optimizing learning, the system comprising:
a vagus nerve stimulator configured to stimulate a vagus nerve of a person with an electrical pulse train;
a controller configured to alter one or more parameters of the electrical pulse train while maintaining other parameters of the electrical pulse train constant; and
a monitoring device configured to monitor skill learning, wherein the controller receives feedback from the monitoring device and alters the parameters of the electrical pulse train of the vagus nerve stimulator.

2. The closed loop system of claim 1 wherein the vagus nerve stimulator is configured to stimulate a vagus nerve of a person with an electrical pulse train prior to the person performing a process related to the skill learning.

3. The closed loop system of claim 1 wherein the vagus nerve stimulator is configured to stimulate a vagus nerve of a person with an electrical pulse train for a period of 1-5 seconds prior to the person performing a process related to the skill learning.

4. The closed loop system of claim 1 wherein the vagus nerve stimulator is configured to stimulate a vagus nerve of a person with an electrical pulse train after the person performs a process related to the skill learning.

5. The closed loop system of claim 1 wherein the vagus nerve stimulator is configured to stimulate a vagus nerve of a person with an electrical pulse train for a period of 1-5 seconds after the person performs a process related to the skill learning.

6. The system of claim 1 wherein:
the one or more parameters of the electrical pulse train altered by the controller include electrical pulse train duration and electrical pulse train frequency; and
the parameters of the electrical pulse train maintained constant include pulses per electrical pulse train, current intensity, and total charge.

7. The system of claim 6 wherein the electrical pulse train duration is altered between 0.125 seconds, 0.5 seconds and 2.0 seconds.

8. The system of claim 6 wherein the electrical pulse train frequency is altered between 7.5 Hz, 30 Hz, and 120 Hz.

9. The system of claim 1 wherein:
the one or more parameters of the electrical pulse train altered by the controller include pulses per electrical pulse train, electrical pulse train frequency, and total charge; and
the parameters of the electrical pulse train maintained constant include current intensity.

10. The system of claim 1 wherein:
the one or more parameters of the electrical pulse train altered by the controller include electrical pulse train duration, pulses per electrical pulse train, and total charge; and
the parameters of the electrical pulse train maintained constant include current intensity and electrical pulse train frequency.

11. The system of claim 1 wherein:
the one or more parameters of the electrical pulse train altered by the controller include current intensity, electrical pulse train frequency, and pulses per electrical pulse train; and
the parameters of the electrical pulse train maintained constant include total charge and electrical pulse train duration.

12. The system of claim 1 wherein the vagus nerve stimulator is an implantable or transcutaneous device.

13. A method of optimizing targeted neuroplasticity training, the method comprising:
stimulating a vagus nerve of a person with an electrical pulse train for a time period before or after the person performs a skill learning process; and
monitoring the skill learning process before and after the vagus nerve is stimulated with the electrical pulse train.

14. The method of claim 13 wherein the time period is 1-5 seconds before the person performs the skill learning process.

15. The method of claim 13 wherein the time period is 1-5 seconds after the person performs the skill learning process.

16. The method of claim 13, further comprising altering one or more parameters of the electrical pulse train while maintaining other parameters of the electrical pulse train constant.

17. The method of claim 16 wherein altering one or more parameters of the electrical pulse train while maintaining other parameters of the electrical pulse train constant comprises:
altering an electrical pulse train duration and an electrical pulse train frequency; and
maintaining constant an electrical pulse train, current intensity, and total charge.

18. The method of claim 16 wherein altering one or more parameters of the electrical pulse train while maintaining other parameters of the electrical pulse train constant comprises:
altering an electrical pulse train, electrical pulse train frequency, and total charge; and
maintaining constant a current intensity.

19. The method of claim 16 wherein altering one or more parameters of the electrical pulse train while maintaining other parameters of the electrical pulse train constant comprises:
altering an electrical pulse train duration, pulses per electrical pulse train, and total charge; and
maintaining constant a current intensity and electrical pulse train frequency.

20. The method of claim 16 wherein altering one or more parameters of the electrical pulse train while maintaining other parameters of the electrical pulse train constant comprises:
altering a current intensity, electrical pulse train frequency, and pulses per electrical pulse train; and
maintaining constant a total charge and an electrical pulse train duration.
